(19) United States
(12) Patent Application Publication
(10) Pub. No.: US 2008/0207711 A1
(43) Pub. Date: Aug. 28, 2008

(54) Method for treating an ID2-related disorder

(76) Inventors: Rong Li, Kansas City, MO (US); Xiaogang Li, Prairie Village, KS (US)

Correspondence Address: Kevin C. Hooper, Esq.;Bryan Cave LLP, 1290 Avenue of the Americas, New York, NY, 10104, US

(21) Appl. No.: 12/072,740

(22) Filed: Feb. 28, 2008

Related U.S. Application Data
(60) Provisional application No. 60/892,136, filed on Feb. 28, 2007.

(51) Int. Cl. A61K 31/425

(52) U.S. Cl. 514/369

(57) ABSTRACT

Described herein is a method of treating an ID2-related disorder comprising administering to a patient in need thereof an ID2 inhibitor or derivative thereof.

### FIELD OF THE INVENTION

The invention relates to methods for treating Id2-related disorders.

### BACKGROUND OF THE INVENTION

Polycystic kidney disease (PKD) is a common inherited human nephropathy that affects 1:1000 of the worldwide population and is the third-most common cause of end stage renal failure. Renal cysts progressively develop in patients afflicted with PKD, and are associated with complications such as cyst infection and hemorrhage, renal stones and pain. PKD largely results from inheriting a single mutant copy of either the Pkd1 or Pkd2 gene. These genes respectively encode the large, membrane-associated proteins Polycystin-1 (PC1) and Polycystin-2 (PC2). Loss of either Pkd1 or Pkd2 function results in hyperproliferation and lack of differentiation of kidney cells.

There is no known treatment to prevent or alleviate the hyperproliferative cell defects associated with PKD. Additionally, theoretical treatments such as chemopreventive therapies currently used to treat other non-PKD-related diseases are fraught with high toxicity and lack of tolerability in patients. Hence, there is a need for preventing and alleviating symptoms of PKD.

### SUMMARY OF THE INVENTION

A method of treating an Id2-related disorder is provided. The method may comprise administering to a patient in need thereof a therapeutically effective amount of an Id2 inhibitor or derivative thereof. The Id2-related disorder may be polycystic kidney disease, or cancer. The polycystic kidney disease may be autosomal dominant polycystic kidney disease or autosomal recessive polycystic kidney disease.

The Id2 inhibitor may be a thiazolidinedione, which may be a glitazone.

The glitazone may have the general formula of:

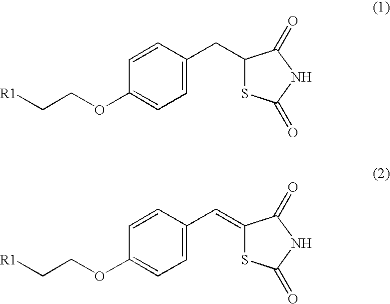

wherein R1 is selected from the group consisting of an aryl group and a substituted aryl group. The R1 substituent group may be also selected from the group consisting of

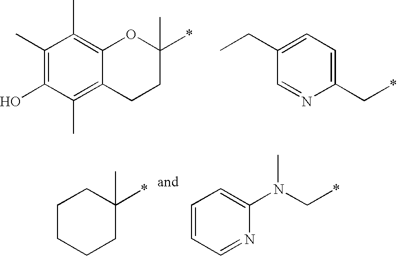

The glitazone selected from the group consisting of (a) rosiglitazone, (b) ciglitazone, (c) pioglitazone, (d) may also be troglitazone, (e) englitazone, and (f) a derivative of (a)-(e). The glitazone may be administered at a dosage in the range of 0.1 mg/kg/day to 1.0 mg/kg/day.

### BRIEF DESCRIPTION OF THE DRAWINGS

FIG. 1. Rosiglitazone inhibits cyst formation in Pkd2^{-/-} kidneys. (a) wild-type and (b) Pkd2^{-/-} kidneys without rosiglitazone treatment. (c) Pkd2^{+/-} and (d) Pkd2^{-/-} kidneys with rosiglitazone treatment.

### DETAILED DESCRIPTION

The inventors have made the surprising discovery that Id2 inhibitors may be used to treat polycystic kidney disease. While not being bound by theory, PKD may be due to increased Id2 activity, which may lead to an increase in cell proliferation and a decrease in cell differentiation. Id2 may decrease activity of the cyclin-dependent kinase inhibitor p21, which, in contrast, may regulate the cell cycle and promote cell differentiation. Polycystin 2 (PC2) may regulate the biological activity of Id2 in the cell cytoplasm. Polycystin 2 is a protein product of the Pkd2 gene. Wild type PC2 sequesters and prevents Id2 from entering the nucleus. Loss of PC2 function may allow increased amounts of Id2 to enter the nucleus, thereby decreasing the level of p21. These increased amounts of Id2 may cause an Id2-related disorder, which in turn, may lead to uncontrolled cell proliferation and lack of cell differentiation that may lead to polycystic kidney disease (PKD). Inhibition of Id2 may control cell proliferation and increase cell differentiation. Id2 inhibitors may be agents from a wide array of different compounds. These insights lead to use of other related Id2-inhibitory agents to treat other diseases affected by Id2-related disorders. Other aspects of the invention will become apparent to the skilled artisan by the following description of the invention.

### 1. Definitions.

The terminology used herein is for the purpose of describing particular embodiments only and is not intended to be limiting. As used in the specification and the appended claims, the singular forms “a,” “an” and “the” include plural referents unless the context clearly dictates otherwise.

“Cancer” as used herein may mean the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, renal, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, teratocarcinoma, renal cell carcinoma (RCC), pancreatic cancer, myeloma, myeloid and lymphoblastic leukemia, neuroblastoma, and glioblastoma.

“Treatment” or “treating,” when referring to protection of an animal from a disease, means preventing, suppressing, repressing, or completely eliminating the disease. Preventing the disease involves administering a composition of the present invention to an animal prior to onset of the disease. Suppressing the disease involves administering a composition of the present invention to an animal after induction of the disease but before its clinical appearance. Repressing the disease involves administering a composition of the present invention to an animal after clinical appearance of the disease.

### 2. Id2 Mechanism

ID2 inhibitors or agents may be used for treating Id2-related disorders by administering to a patient in need thereof. Id2 inhibitors or agents may inhibit cell proliferation, but increase cell differentiation. While not being bound by theory, Id2 may be a protein which is a member of the helix-loop-helix family of transcription factors that promotes cell growth and inhibits differentiation. Id2 may act as a dominant-negative inhibitor of basic helix-loop-helix proteins by inhibiting DNA binding and subsequent transcription activator. Id2 may also play a role in cell cycle control, which is consistent with high Id2 expression observed in proliferating cells and low expression in non-proliferating cells. In the nucleus, Id2 may repress transcription of p21. p21 may inhibit CDK2 activity, which in turn, may prevent cell cycle progression. Hence, p21 may inhibit cell division and promote cell differentiation.

Proper Id2 regulation may be critical in maintaining proper rates of cell division and differentiation. Id2 may be downregulated in differentiated cells, and conversely, increasing Id2 expression may inhibit cell differentiation and increases cell proliferation. Id2 proteins may be sequestered in the cytoplasm by polycystin, which in turn, may regulate the cell cycle. PC2 may be able to interact with Id2 by being phosphorylated by polycystin-1-dependent phosphorylation. Phosphorylated PC2 may suppress Id2 nuclear translocation thereby preventing Id2 from repressing the transcription of p21.

An Id2-inhibitory agent may treat disorders associated with autosomal dominant or autosomal recessive mutations of Pkd1 or Pkd2. Autosomal dominant mutations in Pkd2 result in a PC2 product that is unable to sequester Id2 in the cytoplasm. Enhanced levels of Id2 may translocate to the nucleus and repress transcription of p21, thereby promoting cell growth and inhibiting differentiation. An Id2 inhibitor may decrease the level of Id2 in the cytoplasm to overcome the effects of Pkd1 or Pkd2 loss of function. Decreasing the level of Id2 may also result in an increase in p21 expression, which controls the cell cycle, decreases cell proliferation, and increases cell differentiation.

### 3. Id2-Related Disorder

An Id2-inhibitory agent may be used to treat an Id2-related disorder by decreasing the activity of Id2 in the cell. The Id2-related disorder may be cancer or polycystic kidney disease. Polycystic kidney disease may be autosomal dominant or autosomal recessive polycystic kidney disease.

### 4. Id2 Inhibitor

An agent that inhibits the activity of Id2 may be used to treat an Id2-related disorder by administering the agent. The agent may be administered to a patient in need thereof. The Id2-inhibitory agent may enable the CDK inhibitor p21 to control the cell cycle, prevent cell proliferation, and promote cell differentiation.

The agent may inhibit the activity of Id2 by reducing expression of the Id2 gene. The agent may also reduce the activity of Id2 by contact with the agent. The agent may also increase the activity of p21.

The agent may be a peroxisome proliferator-activated receptor (PPAR)γ agonist. The PPARγ agonist may be a thiazolidinedione, or a derivative thereof. The thiazolidinedione may be a glitazone. The thiazolidinedione derivative may be a Δ2 derivative, which may comprise a double bond adjoining the terminal thiazolidine-2,4-dione ring.

a. Thiazolidinedione

The thiazolidinedione or derivative thereof may be a compound as described in European Patent Publication No. 306228, the contents of which are incorporated herein by reference. The thiazolidinedione may comprise 5-[4-[2-(N-methyl-N-(2-pyridyl)amino)ethoxy]benzyl]thiazolidine-2,4-dione (also known as rosiglitazone). Salts of this compound including the maleate salt are described in International Publication No. WO94105659, the contents of which are incorporated herein by reference. The thiazolidinedione may also comprise a compound as described in European Patent Publication Nos. 0008203, 0139421, 0032128, 0428312, 0489663, 0155845, 0257781, 0208420, 0177353, 0319189, 0332331, 0332332, 0528734, and 0508740; International Patent Publication Nos. 92118501, 93102079, 93122445; or U.S. Pat. Nos. 5,104,888 and 5,478,852, the contents of which are incorporated herein by reference. The thiazolidinedione may comprise 5-[4-[2-(5-ethyl-2-pyridyl)ethoxy]benzyl]thiazolidine-2,4-dione (also known as pioglitazone); 5-[4-[(1 methylcyclohexyl)methoxy]benzyl]thiazolidine-2,4-dione (also known as ciglitazone), 5[[4-[(3,4-dihydro-6-hydroxy-2,5,7,8-tetramethyl-2H-1-benzopyran-2-yl)methoxy]phenyl]methyl]-2,4-thiazolidinedione (also known as troglitazone); or 5-[(2-benzyl-2,3-dihydrobenzopyran)-5-ylmethyl)thiazolidine-2,4-dione (also known as englitazone).

The thiazolidinedione or derivative thereof may be of chemical structure 1 or 2 as follows:

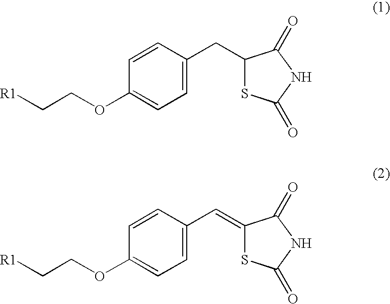

wherein R1 may comprise a chemical structure selected from the group consisting of:

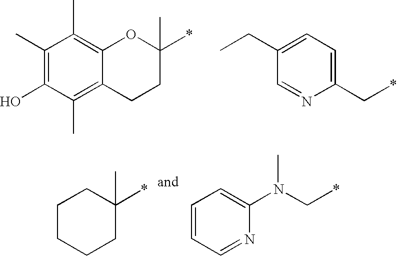

b. Formulation

An Id2-inhibitory agent maybe administered for treating an Id2-related disorder in a number of different formulations. The agent may be formulated as a tablet or lozenge in a conventional manner. For example, tablets and capsules for oral administration may contain conventional excipients such as a binding compound, filler, lubricant, disintegrant or wetting compound. The binding compound may be syrup, accacia, gelatin, sorbitol, tragacanth, mucilage of starch or polyvinylpyrrolidone. The filler may be lactose, sugar, microcrystalline cellulose, maizestarch, calcium phosphate, or sorbitol. The lubricant may be magnesium stearate, stearic acid, talc, polyethylene glycol, or silica. The disintegrant may be potato starch or sodium starch glycollate. The wetting compound may be sodium lauryl sulfate. Tablets may be coated according to methods well known in the art.

The agent may also be a liquid formulation which may be an aqueous or oily suspension, solution, emulsion, syrup, or elixir. The agent may also be formulated as a dry product for constitution with water or other suitable vehicle before use. Such liquid preparations may contain an additive such as a suspending compound, emulsifying compound, nonaqueous vehicle or preservative. The suspending compound may be a sorbitol syrup, methyl cellulose, glucose/sugar syrup, gelatin, hydroxyethylcellulose, carboxymethyl cellulose, aluminum stearate gel, or a hydrogenated edible fat. The emulsifying compound may be lecithin, sorbitan monooleate, or acacia. The nonaqueous vehicle may be an edible oil, almond oil, fractionated coconut oil, oily ester, propylene glycol, or ethyl alcohol. The preservative may be methyl or propyl p-hydroxybenzoate, or sorbic acid.

The agent may also be formulated as a suppository, which may contain suppository bases which may be cocoa butter or glycerides. The agent may also be formulated for inhalation, which may be in a form such as a solution, suspension, or emulsion that may be administered as a dry powder or in the form of an aerosol using a propellant, such as dichlorodifluoromethane or trichlorofluoromethane. The agent may also be a transdermal formulation comprising an aqueous or nonaqueous vehicle which may be a cream, ointment, lotion, paste, medicated plaster, patch, or membrane.

The agent may also be formulated for parenteral administration which may be by injection or continuous infusion. Formulations for injection may be in the form of a suspension, solution, or emulsion in oily or aqueous vehicles, and may contain a formulation compound such as a suspending, stabilizing, or dispersing compound. The agent may also be provided in a powder form for reconstitution with a suitable vehicle such as sterile, pyrogen-free water.

The agent may also be formulated as a depot preparation, which may be administered by implantation or by intramuscular injection. The agent may be formulated with a suitable polymeric or hydrophobic material (as an emulsion in an acceptable oil, for example), ion exchange resin, or as a sparingly soluble derivative (as a sparingly soluble salt, for example).

The agent may also be formulated as a liposome preparation. The liposome preparation may comprise a liposome which penetrates the cells of interest or the stratum corneum, and fuses with the cell membrane, resulting in delivery of the contents of the liposome into the cell. For example, the liposome may be as described in U.S. Pat. No. 5,077,211 of Yarosh, U.S. Pat. No. 4,621,023 of Redziniak et al. or U.S. Pat. No. 4,508,703 of Redziniak et al., the contents of which are incorporated herein by reference. The agent may be intended to target a skin condition and may be administered before, during, or after exposure of the skin of a mammal to an UV or oxidative damage-causing compound. Other suitable formulations may employ niosomes. Niosomes are lipid vesicles similar to liposomes, with membranes consisting largely of non-ionic lipids, some forms of which are effective for transporting compounds across the stratum corneum.

c. Administration of Agent

An Id2 inhibitory agent may be administered orally, parenterally, sublingually, transdermally, rectally, transmucosally, topically, via inhalation, via buccal administration, or combinations thereof. Parenteral administration may be intravenous, intraarterial, intraperitoneal, subcutaneous, intramuscular, intrathecal, or intraarticular. The agent may also be administered in the form of an implant, which allows slow release of the agent as well as a slow controlled i.v. infusion.

d. Dosage of Agent

A therapeutically effective amount of the agent required for use in therapy varies with the nature of the condition being treated, the length of time that activity is desired, and the age and the condition of the patient, and ultimately may be determined by the attendant physician. In general, however, doses employed for adult human treatment typically may be in the range of 0.001 mg/kg to about 200 mg/kg per day. The dosage may be about 1 mg/kg a day to about 100 mg/kg per day. The dosage may be 2, 3, 4, 5, 6, 7, 8, 9, 10 to 50, 60, 70, 80, 90, 100 mg/kg per day. The dose may be about 1 μg/kg to about 100 μg/kg per day. The desired dose may be conveniently administered in a single dose, or as multiple doses administered at appropriate intervals, for example as two, three, four or more subdoses per day. Multiple doses often are desired, or required.

A number of factors may lead to the agent being administered at a wide range of dosages. When given in combination with other therapeutics, the dosage of the agent of the present invention may be given at a relatively lower dosage. In addition, the use of a targeting substituent may allow the necessary dosage to be relatively low. The agent may be administered at a relatively high dosage, which may be due to a factor such as low toxicity, high clearance, or low rates of processing. As a result, the dosage of the agent may be from about 1 ng/kg to about 100 mg/kg. The dosage of the agent may be at any about 1 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, or 100 mg/kg.

The present invention has multiple aspects, illustrated by the following non-limiting examples.

### EXAMPLE 1

### Reduced Id2 Levels Diminish PKD Phenotypes

Reduced Id2 gene levels diminish Pkd gene loss of function phenotypes. This was shown by observing kidney size and the amount of kidney cysts in Pkd^{-/-} mutant mice with varying Id2 genotypes, and therefore different doses of functional Id2 gene. Pkd2^{-/-} Id2^{-/-} double mutant mice were generated by inter-crossing Pkd2^{+/-} Id2^{+/-} mice. Pkd2^{-/-} Id2^{+/+} littermates exhibited cyst-filled kidneys that were much larger than wild-type kidneys. Additionally, these mice displayed severe edema, which was likely due to kidney failure. In contrast, newborn Pkd2^{-/-} Id2^{+/-} mice, which had half the Id2 gene copy number of Pkd2^{-/-} Id2^{+/+} littermates, kidneys, exhibited some cysts, but their kidneys were only slightly larger than in wild-type. Further, Pkd2^{-/-} Id2^{-/-} double mutant mice displayed no edema. Therefore, decreasing the gene dose level of Id2 decreases Pkd loss of function phenotype, suggesting that (1) Id2 plays a role in mediating the Pkd mutant phenotype and (2) inhibiting Id2 activity may alleviate effects of Pkd mutations.

### EXAMPLE 2

### Rosiglitazone Treats PKD in Mice

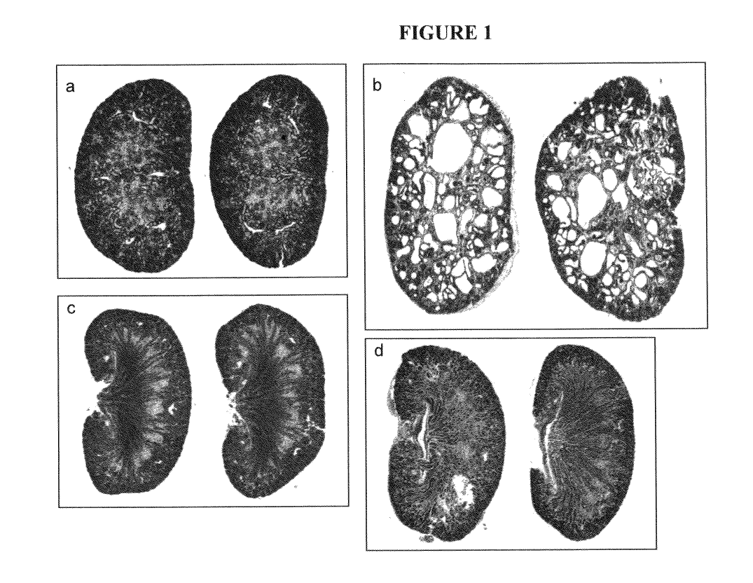

Rosiglitazone, which may inhibit Id2 activity, alleviates the effects of Pkd loss of function mutations. This was shown by treating pregnant female mice from a cross between Pkd2^{+/-} mice with 80 mg/kg diet/day ciglitazone or rosiglitazone for ten days prior to delivery. Consistent with the predicted effects of an Id2 inhibitor, rosiglitazone treatment resulted in small body size in six of newborn pups. Similarly, Id2 knockout mice were small. Significantly, a Pkd2^{-/-} pup born to a rosiglitazone-treated mother had kidneys of normal size and morphology (FIG. 1d) compared to its Pkd2^{-/-} littermates born to untreated mothers (FIG. 1b), and compared to its Pkd2^{+/+} (FIG. 1a) and Pkd2^{+/-} (FIG. 1c) littermates. Furthermore, the Pkd2^{-/-} pup born to a rosiglitazone-treated mother exhibited no edema, indicating functioning kidneys. In contrast, ciglitazone treatment appeared to have no effect on newborn Pkd2^{-/-} mice. Hence, rosiglitazone appears to be capable of reducing the symptoms of Pkd mutations, possibly by inhibiting Id2.

1. A method of treating an Id2-related disorder comprising administering to a patient in need thereof a therapeutically effective amount of an Id2 inhibitor or derivative thereof.

2. The method of claim 1, wherein the Id2-related disorder is selected from the group consisting of PKD, diabetes, and cancer.

3. The method of claim 1, wherein the Id2-related disorder is PKD.

4. The method of claim 3, wherein the PKD is ADPKD or ARPKD.

5. The method of claim 1, wherein the Id2 inhibitor is thiazolidinedione.

6. The method of claim 1, wherein the thiazolidinedione is a glitazone.

7. The method of claim 6, wherein the glitazone has the general formula selected from the group consisting of: 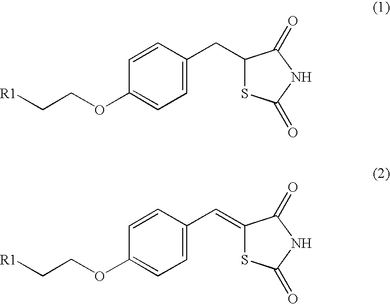
wherein R1 is selected from the group consisting of an aryl group and a substituted aryl group.

8. The method of claim 7, wherein the R1 group is selected from the group consisting of:

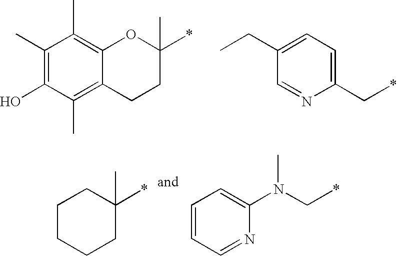

9. The method of claim 7, wherein the glitazone is selected from the group consisting of: (a) rosiglitazone, (b) ciglitazone, (c) pioglitazone, (d) troglitazone, (e) englitazone and (f) a derivative of (a)-(e).

10. The method of claim 1, wherein Id2 activity is decreased compared to a control.

11. The method of claim 10, wherein the decreased Id2 activity is due a decrease in gene expression of Id2 in comparison to a control.

12. The method of claim 9, wherein the glitazone is rosaglitazone.

13. The method of claim 12, wherein rosaglitazone is administered at a therapeutically effective dosage range between 1 mg/kg/day to 100 mg/kg/day.

14. The method of claim 12, wherein rosaglitazone is administered at a therapeutically effective dosage range between 10 to 80 mg/kg/day.
